(19) United States
(12) Patent Application Publication
(10) Pub. No.: US 2018/0325797 A1
(43) Pub. Date: Nov. 15, 2018

(54) Edible Deodorant Wipe, Method of Making and Method of Using

(71) Applicant: Sheena R. Despert, Pittsburgh, PA (US)

(72) Inventor: Sheena R. Despert, Pittsburgh, PA (US)

(21) Appl. No.: 15/975,491

(22) Filed: May 9, 2018

Related U.S. Application Data
(60) Provisional application No. 62/503,620, filed on May 9, 2017.

(51) Int. Cl. A61K 8/73; A61K 8/92; A61K 8/67; A61K 8/19; A61K 8/02; A61Q 15/00

(52) U.S. Cl. CPC A61K 8/732; A61K 8/922; A61Q 15/00; A61K 8/19; A61K 8/0208; A61K 8/678

(57) ABSTRACT

The present invention discloses a hygienic wipe product that includes an “edible” cleansing composition containing arrowroot, baking soda, virgin coconut oil, Shea butter, tea tree oil, essential oils, vitamin E and apple vinegar.

### CROSS-REFERENCE TO RELATED APPLICATIONS

This is a perfection of U.S. Provisional Application Ser. No. 62/503,620, filed on May 9, 2017, the disclosure of which is fully incorporated by reference herein.

### BACKGROUND OF INVENTION

This invention is directed to a cleansing composition that is intended for use with disposable tissue products or wipes. More particularly, the present invention is directed to a novel composition for cleaning the sensitive areas of a male or female, such as the genitals and/or rectal regions, without causing any risk of negative health effects if contacted by mouth, or accidentally swallowed.

The perianal, uro-genital, and vaginal regions of an adult are especially vulnerable to personal hygiene concerns. Cleansing of these areas should be a prerequisite before any sexual intimacy is undertaken. Yet, it would be undesirable to use as a wipe or disposable towel/tissue something which is either harmful if contacted by mouth, OR is otherwise “unpalatable”, i.e., tastes bad.

Based on the foregoing, there is a need for a product such as a towelette that can gently clean these sensitive areas of the human body without imparting damage thereto or otherwise detracting from the start of some intimacy between consenting adults.

There are several known uses of arrowroot in numerous deodorant-related inventions and the like. They include the disclosures at: U.S. Published Application No. 20050002872, U.S. Published Application No. 20050129741, U.S. Published Application No. 20060134045, U.S. Published Application No. 20070202062 and U.S. Pat. Nos. 6,299,867, 8,663,611 and 9,084,764 just to name a few.

### SUMMARY OF THE INVENTION

The present invention is directed to a composition for use with tissue products such as a disposable wipe or washcloth. The composition comprises a novel combination of arrowroot starch, baking soda, various oils and Shea butter, with apple vinegar, vitamin E and a preferred combination of essential oils (for scent disparity). The aforesaid combinations are mixed together in a preferred order (for a so-called “recipe”), then poured over new, sterile wipe products before being allowed to ‘set’ via refrigeration. This invention also addresses a method for using the aforementioned composition-containing wipes to achieve greater hygiene in various regions of the human body prior to the start of intimacy. They can also be used for cleaning up afterwards.

### DESCRIPTION OF PREFERRED EMBODIMENTS

The present invention is described in relation to a cleansing solution for a disposable wipe or other (paper) tissue product. The composition itself is suitable for use on one or both surfaces of that tissue product. On a preferred basis, such tissue products like bath tissue, facial tissue, disposable towels, napkins, hankies, and/or polyolefin wipes, can be held in a dispensing container (such as a diaper wipes “tub”) in which some additional composition may be included for wicking up into the wipes over time.

The paper products suitable for use with this cleansing composition can be homogenous or multi-layered, in a single- or multi-ply construction. Conventionally pressed tissue paper and methods for making such paper are well known in the art. Such paper is typically made by depositing a papermaking furnish on a foraminous forming wire, often referred to in the art as a Fourdrinier wire. Once the furnish is deposited on the forming wire, it is referred to as a web. That web is then dewatered by pressing and drying at elevated temperatures.

The particular techniques and typical equipment for making webs according to the process just described are also well known in the art. In a typical process, a low consistency pulp furnish is provided from a pressurized headbox with an opening for delivering a thin deposit onto Fourdrinier wire to form a wet web. That web is then typically dewatered by vacuum dewatering and further dried by pressing particularly when the web is subjected to pressure from opposing mechanical members or cylindrical rolls. These formed sheets are considered to be compacted since the entire web gets subjected to substantial mechanical compressional forces while the fibers are moist, then dried while still in a compressed state.

The cleansing formulation described herein for use in combination with the aforementioned “family” of tissue products should impart a soft, lubricious, lotion-like feel to the touch. The cleansing formulation gently removes microbes and soils upon use to improve the overall health AND hygiene of its user(s).

In one embodiment of the present invention, the cleansing composition includes a water soluble starch, more particularly arrowroot, in a compositional range between about 30-40%, by weight. Preferred levels of arrowroot addition range between about 32-37% with a compositional addition of about 35% being most preferred.

In one suitable embodiment, the aforementioned arrowroot starch product, as a powder, is combined with about 20-30% baking soda so that when these two main additives are combined, they make up over 50% of the composition proper. Preferably, the final composition contains about 22-27% baking soda and most preferably about 25% by weight.

In order of percentages, the next main additive for this “edible” formulation is about 10-18% virgin coconut oil, preferably about 12-16% and most preferably 14% by weight.

The composition has as its fourth most prevalent additive about 7-12% Shea butter, more preferably about 10% by weight. To that “mix”, one should include about 5-10% tea tree oil, more preferably about 7% by weight.

To all of the foregoing, one should add about 3-7% essential oils, more preferably about 5 wt. %, with a combination of the same selected from (for scent and/or “flavoring” purposes): one or more of lemon, lavender, grapefruit, eucalyptus, cedar wood and/or peppermint.

Critical to the aforementioned, one should include about 1-3 wt. % vitamin E, preferably about 2% by weight; AND about 1-3 wt. % apple vinegar, also more preferably about 2% by weight.

A method of making the aforementioned cleansing composition entails the following preferred steps: (1) melt the coconut oil to a liquid state; (2) add to the melted coconut oil the powdered versions of both arrowroot and baking soda; (3) after the Shea butter has been melted, separately, it can be added; (4) the various oils, vitamin E and apple vinegar are then combined with the aforementioned and poured over a collection of wrapped sanitary wipes, or towelettes. This “combination” of cleansing solution and disposable tissues is then refrigerated to ‘set’ the components into the wiping towels. It results in a hygienic wipe that would NOT prove harmful if accidentally swallowed or otherwise brought in contact with a human's mouth and/or private parts.

A method of using the novel composition described above would entail; (a) providing a package (or other collection) of such towelettes that have been covered with this cleansing composition . . . and possibly stored with some additional solution for keeping the towelettes sufficiently moist before use; (b) removing one or more towelettes from their holding container; (c) wiping the sensitive areas of the human body with the removed towelettes; and (d) properly disposing of the used towelettes thereafter.

Although intended as a hygienic wipe for the private areas of the human body prior to intimate relations, it is to be understood that these same wipes could be used as a deodorant-style wipe to one's underarms, knees and around one's neck.

In view of the above, it will be seen that the several objects of the invention are achieved. As various changes could be made in the above-described products and methods without departing from the scope of the invention, it is intended that all matter contained in the above description be interpreted as illustrative and not in a limiting sense.

What is claimed is:

1. A hygiene wipe composition comprising: about 30-40 wt. % arrowroot, about 20-30 wt. % baking soda, about 10-18 wt. % virgin coconut oil, about 7-12 wt. % Shea butter, about 5-10 wt. % tea tree oil, about 3-7 wt. % essential oils, about 1-3 wt. % vitamin E, and about 1-3 wt. % apple vinegar.

2. The wipe composition of claim 1, which contains about 35 wt. % arrowroot.

3. The wipe composition of claim 1, which contains about 25 wt. % baking soda.

4. The wipe composition of claim 1, which contains about 14 wt. % virgin coconut oil.

5. The wipe composition of claim 1, which contains about 10 wt. % Shea butter.

6. The wipe composition of claim 1, which contains about 7 wt. % tea tree oil.

7. The wipe composition of claim 1, which contains about 5 wt. % essential oils.

8. The wipe composition of claim 1 wherein the essential oils include one or more of lemon, lavender, grapefruit, eucalyptus, cedar wood and peppermint.

9. The wipe composition of claim 1, which contains about 2 wt. % vitamin E.

10. The wipe composition of claim 1, which contains about 2 wt. % apple vinegar.

11. The wipe composition of claim 1, which is applied to a paper product selected from the group consisting of bath tissue, facial tissue, disposable towels, napkins, hankies, and polyolefin wipes.

12. A method of making a disposable hygienic wipe comprises: (a) providing a set of ingredients that includes arrowroot, baking soda, virgin coconut oil, Shea butter, tea tree oil, essential oils, vitamin E and apple vinegar; (b) melting the coconut oil to a liquid state; (c) adding to the melted coconut oil the powdered versions of both arrowroot and baking soda; (d) melting the Shea butter and adding it to the aforementioned mix has been melted separately, it can be added to the aforementioned; (e) adding in the various oils, vitamin E and apple vinegar; (f) pouring the mix over a collection of wrapped sanitary wipes, or towelettes; and (g) refrigerating the wipes.

13. The method of claim 12 wherein the ingredients consist of about 30-40 wt. % arrowroot, about 20-30 wt. % baking soda, about 10-18 wt. % virgin coconut oil, about 7-12 wt. % Shea butter, about 5-10 wt. % tea tree oil, about 3-7 wt. % essential oils, about 1-3 wt. % vitamin E, and about 1-3 wt. % apple vinegar.

14. A method for cleaning sensitive areas of the human body before intimacy, said method comprising: (a) providing a package of towelettes that have been soaked in a cleansing composition comprising: about 30-40 wt. % arrowroot, about 20-30 wt. % baking soda, about 10-18 wt. % virgin coconut oil, about 7-12 wt. % Shea butter, about 5-10 wt. % tea tree oil, about 3-7 wt. % essential oils, about 1-3 wt. % vitamin E, and about 1-3 wt. % apple vinegar; (b) removing one or more towelettes from the package; (c) wiping the sensitive areas of the human body with the removed towelettes; and (d) disposing the used towelettes.

15. The method of claim 14 wherein said cleansing composition includes: about 35 wt. % arrowroot, about 25 wt. % baking soda, about 14 wt. % virgin coconut oil, about 10 wt. % Shea butter, about 7 wt. % tea tree oil, about 5 wt. % essential oils, about 2 wt. % vitamin E, and about 2 wt. % apple vinegar.

16. The method of claim 14 wherein the essential oils include one or more of lemon, lavender, grapefruit, eucalyptus, cedar wood and peppermint.
